(19) United States
(12) Patent Application Publication
(10) Pub. No.: US 2021/0008252 A1
(43) Pub. Date: Jan. 14, 2021

(54) THERMOPLASTIC MATERIALS INCORPORATING BIOACTIVE INORGANIC ADDITIVES

(71) Applicant: Evonik Operations GmbH, Essen (DE)

(72) Inventors: Balaji Prabhu, Hoover, AL (US); Mahrokh Dadsetan, Birmingham, AL (US); Subhadip Bodhak, Birmingham, AL (US); Andreas Karau, Gelnhausen (DE); Marc Knebel, Heddesheim (DE); Rosario Lizio, Dieburg (DE); Samuel von Karsa-Wilberforce, Sinzig (DE)

(21) Appl. No.: 17/040,190

(22) PCT Filed: Mar. 25, 2019
(86) PCT No.: PCT/EP2019/057400

§ 371 Date: Sep. 22, 2020

Related U.S. Application Data
(60) Provisional application No. 62/648,378, filed on Mar. 26, 2018.

(51) Int. Cl. A61L 27/46; A61L 27/56; A61L 27/42; C08L 71/00

(52) U.S. Cl. CPC A61L 27/46; A61L 27/56; A61L 2430/12; C08L 71/00; A61L 2430/02; A61L 27/425

(57) ABSTRACT

Composite materials comprising thermoplastic polymeric material such as polyaryletherketones (PAEKs) and inorganic additive species serving to increase the processing and resultant mechanical, thermal, and biological properties of said thermoplastic polymeric material which may be subsequently used in various medical applications after the two materials are mixed through thermal processing methods. The inorganic additive species may be a calcium salt, and may include fluorine ions.

### FIELD OF THE INVENTION

The present invention relates to compositions containing thermoplastic polymeric material as the major component with a secondary phase being comprised of inorganic additive fillers in the form of particles, the combination of which can be subsequently implemented in the manufacture of medical implants or parts thereof.

### BACKGROUND

The use of bioactive materials in orthopaedic implant materials is known. The introduction of bioactive additives to materials can impart a biological response from normally bioinert materials. Increasing the concentration of these bioactive additives can increase the biological response but also may decrease the processability and mechanical properties of the material.

Numerous materials have been described for the preparation of medical implants that possess the required mechanical and biological properties to achieve effective treatment. For instance, metallic-based materials, such as titanium, have seen use in orthopaedic implant applications due to their ability to initiate rapid bone growth as well as their mechanical robustness.

Polymer-based medical implants have seen increased interest over typically chosen metallic implants due to the advantageous properties of polymer-based implants. For instance, the implantation of a polymer-based device is beneficial for the adjacent bone structure as the common problem of stress-shielding associated with the elevated stiffness of metallic implants is alleviated. Further, the polymer-based implants are transparent to X-rays allowing for more thorough examination after the implant has been placed when compared to metallic, radiopaque materials.

PAEK-based implants, specifically polyetheretherketone (PEEK), have been frequently used in orthopaedic medical applications particularly in the form of implants which are inserted adjacent to bone structure. Although the mechanical properties of PAEK materials along with their chemical resistivity and biostability, defined as the ability to not be broken down or degraded in vivo, the inability of PAEK material to initiate a positive biological response, such as bone growth, is known with common biological response begin the growth of fibrous tissue on the material leading to subsidence of the implant.

In order to impart a degree of bioactivity, compounding PAEK materials with bioactive additives to fabricate composite materials, defined as the addition of a secondary material to the polymeric material, has seen recent activity. When added to the biostable PAEK material, these bioactive additives are able to initiate a positive biological response, such as bone growth, upon implantation. With increasing the concentration of these additives within the PAEK material, the bioactivity is increased. In the area of orthopaedic implants, these additives are typically chosen to have a composition which mimics the composition of native bone tissue so as to promote a natural response upon implantation.

However, the processing and resultant mechanical properties of the PAEK material is changed upon the addition of these secondary materials. For instance, addition of a secondary material decreases the flowability of the blend and therefore increases the shear forces required to achieve adequate mixing of the secondary material throughout the polymeric material. This results in the production of agglomerations of the secondary material within the polymeric material matrix.

These agglomerations not only further increase the difficulty of processing the composite material, they also reduce the overall mechanical properties of the composite material. For instance, a commonly used method in the processing of such composites is twin-screw extrusion, wherein co-rotational screws serve to mix the melted polymeric material with the secondary additive in an effort to homogenously distribute the additive throughout the polymer matrix. With increasing additive concentration, interfacial reactions between the polymeric matrix and the additive surface can inhibit the functioning of the extruder.

For example, WO2015/092398 teaches a polymeric material which is preferably polyetheretherketone, and an apatite, for example a hydroxy-containing apatite for use as a dental implant. However, it does not teach the use of such material for increased flowability, or a critical particle size range of the additive for improved flowability

A composite capability of achieving the flow properties that meet or exceed the virgin polymeric material is still needed, and would therefore offer superior processing advantages resulting in improved properties. These attributes are critical in commercial thermal processing such as compounding, extruding, molding and additive manufacturing of medical device rods, stock shapes and or final medical device implant for use in orthopaedic, spine and dental applications which are prone to brittle failure due to the reinforcement of calcium phosphate based additives in increasing concentrations. These additives and additive blends in varying proportions also shows properties can be superior to known prior art and also the starting virgin polymer matrix.

### BRIEF SUMMARY OF THE INVENTION

It is an object of the presently disclosed invention to address the problems associated with the manufacture and resultant poor physical properties and difficulties in processability of polymeric materials blended with inorganic additives which typically pose a challenge when using conventional processing methods.

It is also an object of the presently disclosed invention to utilize combinations of ceramic inorganic additives to address the poor processability and poor mechanical properties of polymeric materials blended with inorganic additives in the formation of composite materials.

Compositions of osteoconductivity composite materials with increased mechanical properties and processability wherein the bioactive additives within the composite are particulate in geometry comprising an average diameter between about 0.5 μm to about 5 μm wherein the polymeric material matrix comprises a powder before processing comprising an average diameter of about 500 μm to about 2500 μm.

It is preferred that the polymer powder and additive particulate be physically blended before melt processing. It is also preferred that the physically blended powder and particulate be dried before processing.

The compositions may be used, for example, to form shaped articles and orthopaedic implants where enhanced bioactivity, ductility, and strength are required. The herein disclosed compositions also aid in the processability of such composites.

It can thus be understood from this teaching that the presently disclosed invention achieves novel compositions capable of enhancing the processability, shown by the flowability of the composites, as well as the mechanical and biological properties of composites.

The present invention describes a composition with novel properties in regards to strength, ductility, and flowability for the processing. This processing may be carried out in order to form, as an example but not a limitation, of orthopaedic medical devices such as, but not limited to, synthetic bone prosthesis, spinal cages, suture anchors, hip, knee implants etc.

### BRIEF DESCRIPTION OF THE DRAWINGS

FIG. 1 depicts a SEM of a sample of fluorinated hydroxyapatite (FHA), β-tricalcium phosphate (β-TCP), and biphasic calcium phosphate (BCP) powder before processing with average particle size frequency distribution plots for the same.

FIG. 2 depicts the effect of the herein disclosed compositions containing on the melt volume flow rate (MVR) of the herein disclosed composites.

FIG. 3 depicts the effect of the herein disclosed compositions containing on the elongation at break (ductility) of herein disclosed the composites.

FIG. 4 depicts the effect of the herein disclosed compositions containing on the tensile strength of herein disclosed the composites.

FIG. 5 depicts the effect of the herein disclosed compositions containing on the flexural strength of herein disclosed the composites.

FIG. 6 depicts the effect of the herein disclosed compositions containing on the notched impact strength of herein disclosed the composites.

FIG. 7 depicts the effect of the herein disclosed compositions containing on the tensile modulus of herein disclosed the composites.

FIG. 8 depicts the effect of the herein disclosed compositions containing on the flexural modulus of herein disclosed the composites.

FIG. 9A depicts that flexural bars fabricated through fused filament fabrication using PEEK Composite Filament containing 20% BCP has a flexural strength comparable to injection molded PEEK Composite specimens comprising the same composition.

FIG. 9B depicts that flexural bars fabricated through fused filament fabrication using PEEK Composite Filament containing 20% BCP has a 10% increase in flexural modulus compared to injection molded PEEK Composite specimens comprising the same composition.

FIG. 10A depicts that tensile bars fabricated through fused filament fabrication using PEEK Composite Filament containing 20% BCP has a tensile strength comparable to injection molded PEEK Composite specimens comprising the same composition.

FIG. 10B depicts that flexural bars fabricated through fused filament fabrication using PEEK Composite Filament containing 20% BCP has a tensile modulus within 15% of injection molded PEEK Composite specimens comprising the same composition.

FIG. 11 depicts that spinal interbody fusion cages fabricated through fused filament fabrication using PEEK Composite Filament containing 20% BCP has a 6% increase in elastic modulus under compressive testing when compared to machined devices.

FIG. 12A depicts pore sizes of the samples.

FIG. 12B depicts that “Mixed pores” contain a combination of “Small” and “Large” pores with increasing pore size moving vertically from the build surface. Cells maintained their viability on the surface of 3D Printed PEEK Composite similar to injection molded PEEK Composite surfaces. The 3D Printed PEEK Composite surface provided more surface area for cell growth.

### DETAILED DESCRIPTION OF THE INVENTION

For the purposes of promoting an understanding of the principles of the invention disclosed herein and not as a limitation of potential embodiments of the herein disclosed invention, reference will be made to specific embodiments with specific language being used to describe the same. It is therefore to be understood that no limitation of the scope of the herein disclosed invention shall be read into the preferred embodiments herein described or further modifications of the disclosed invention, with such further modifications and/or applications being those which would occur normally to one skilled in the art of the herein disclosed invention and related fields of study.

The present invention relates to polymer composite compositions and their enhanced mechanical, biological, and processing properties. In certain embodiments of the invention, the compositions include a homogenous mixture of a biocompatible polymeric material and a bioactive additive, wherein said bioactive additive is able to improve the flowability of the composite. In further preferred embodiments, the additive is embedded or otherwise dispersed in a polymer matrix through thermal processing.

The biocompatible polymer, such as a high-viscosity, high-strength polymer, may be obtained from synthetic or natural sources. The biocompatible polymer may be selected such that it will act to add robustness to the composite and, for example, increase the load-bearing capability. Further, the biocompatible polymer may comprise a bioresorbable or non-bioresorbable polymer. Examples, but not limitations, of preferred non-bioresorbable polymers include polyaryletherketones, such as polyetherketoneketone, polyetherketone, and polyetheretherketone.

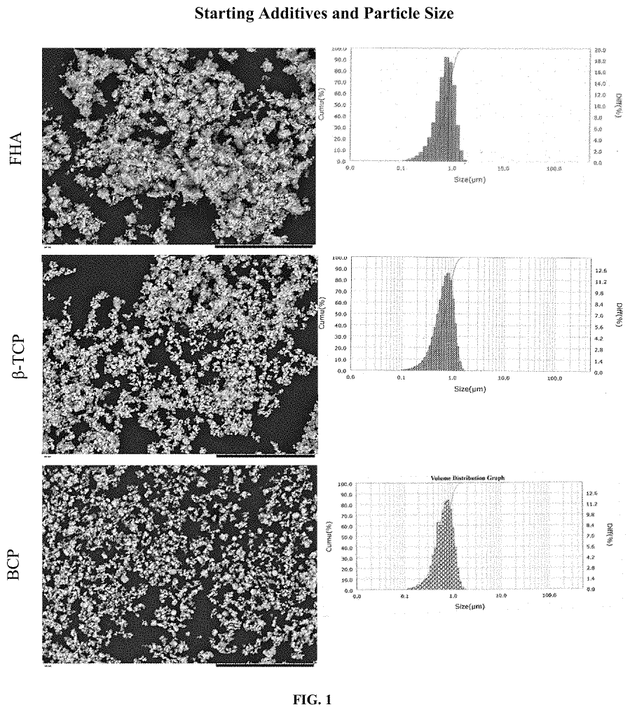

In one aspect of the invention, a composition is provided which is a composite including a homogenous dispersion of a bioactive additive within a biocompatible polymer matrix such as, but not limited to, commercially available VESTAKEEP AR1176 from Evonik Industries. The components are sufficiently mixed together such that the additive is evenly dispersed throughout the polymer matrix with a minimal amount of additive agglomerations. The average particle size of the bioactive additive is favorably not less than about 0.5 μm and not more than about 5 μm. Preferably, additive size does not exceed about 2.5 μm. Further, preferred sizes include an average particle size of not more than about 1.0 μm and most favorably not more than about 0.75 μm. Table 1 depicts the particle size distribution of the additives. The additive size FIG. 1 and molecular makeup may advantageously promote dispersion as well as interfacial reactions between the two phases for improved mechanical, biological, and processing properties, namely, flowability. Said compositions may be advantageously used to form a shaped article useful in orthopaedic applications such as, but not limited to, synthetic bone grafts, spinal cages, and suture anchors. For example, the enhanced flowability of the herein disclosed compositions may be advantageously used to extrude stock-shapes for subsequent machining of a load-bearing article, such as an intervertebral cage, a bone screw, such as interference screws and suture anchors, fixation plates, such as radial fracture plates, and various other orthopaedic implant articles.

TABLE 1

| Additive | D10 | D50 | D90 |
|---|---|---|---|
| Hydroxyapatite | 0.42 um | 0.83 um | 1.3 um |
| Biphasic Calcium Phosphate | 0.35 um | 0.68 um | 1.04 um |
| Fluorapatite | 0.38 um | 0.76 um | 1.21 um |
| B-Tricalcium Phosphate | 0.37 um | 0.69 um | 1.05 um |

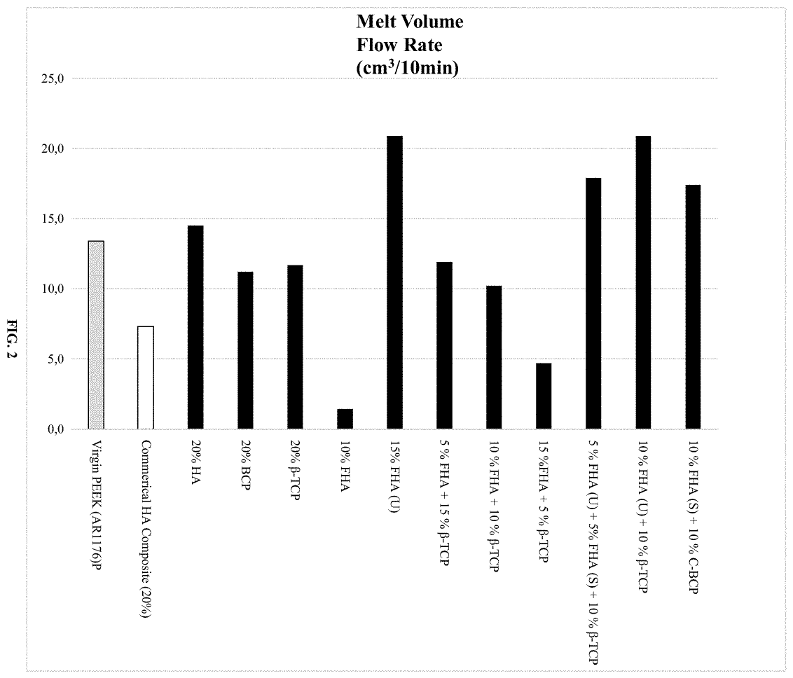

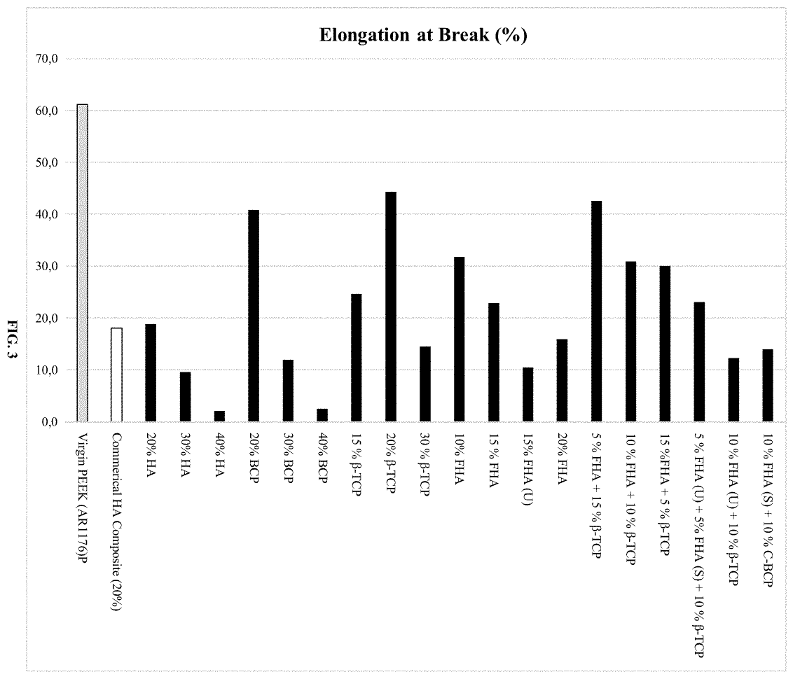

Further forms of the herein disclosed invention require the optimization of injection molding parameters for achieving shaped articles. For example, the optimization of injection molded dog-bone samples was required and necessitated the increase in temperature from the normal operating range of about 380±5° C. to about 410±5°C. to avoid short-shot samples and produce full dog-bone samples. Further, the presently disclosed invention provides inventive compositions for improving the processability, defined herein as flowability, of the composite compositions. Referring now to FIG. 2, the melt flow volume rate (MVR) is improved upon addition of the additive species compared to similar commercially available composites. For example, referring again to FIG. 2, the introduction biphasic calcium phosphate to the polyetheretherketone (PEEK) material results in a melt volume flow rate (MVR) comparable to the virgin polyetheretherketone (PEEK) material containing no additive. Further, referring again to FIG. 2, incorporation of β-tricalcium phosphate (β-TCP) alone has a melt flow volume rate (MVR) comparable to the virgin polyetheretherketone (PEEK) material. Also represented by FIG. 2, the addition of fluorinated hydroxyapatite which is unsintered shows melt volume flow rates superior to that of the virgin polyetheretherketone (PEEK) material. Referring again to FIG. 2, the blend containing β-tricalcium phosphate (β-TCP) and fluorinated hydroxyapatite (FHA) is capable of producing melt volume flow rates comparable to and superior over the virgin polyetheretherkeonte (PEEK) material. The melt volume flow rates (MVR) which are elevated above the virgin polyetheretherketone (PEEK) material may be a result of the effect of particle size FIG. 1 and subsequent homogenous dispersion causing beneficial interfacial reactions between the additive and the polymeric matrix. Specifically, it can be understood that in this example, the β-tricalcium phosphate acts as a flowing aid which may be due to ionic or otherwise occurring interactions between the β-tricalicum phosphate and the fluorinated hydroxyapatite within the polymer matrix.

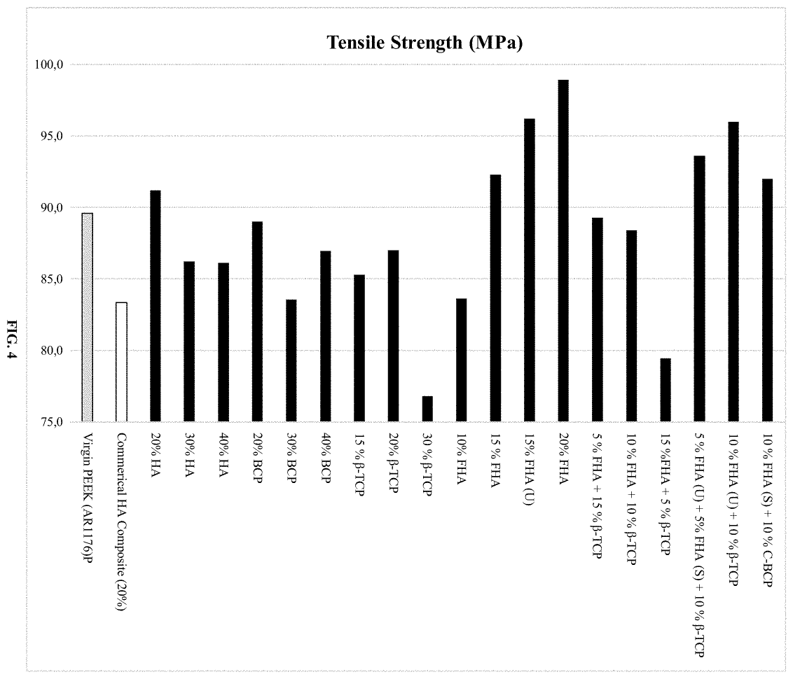

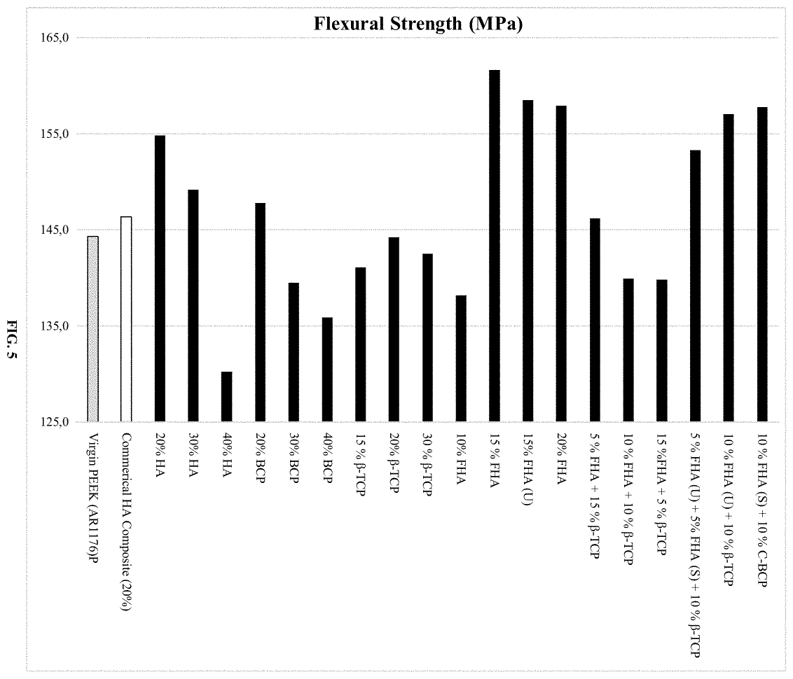

Further embodiments of the presently disclosed invention may require a composite material with elevated mechanical properties such as tensile strength, flexural strength, and notched impact strength. Referring now to FIG. 4, FIG. 5, and FIG. 6, it is understood that the presently disclosed invention is capable of producing composite materials with tensile strength, flexural strength, and notched impact strength superior to both the virgin polyetheretherketone (PEEK) and similar commercially available composites.

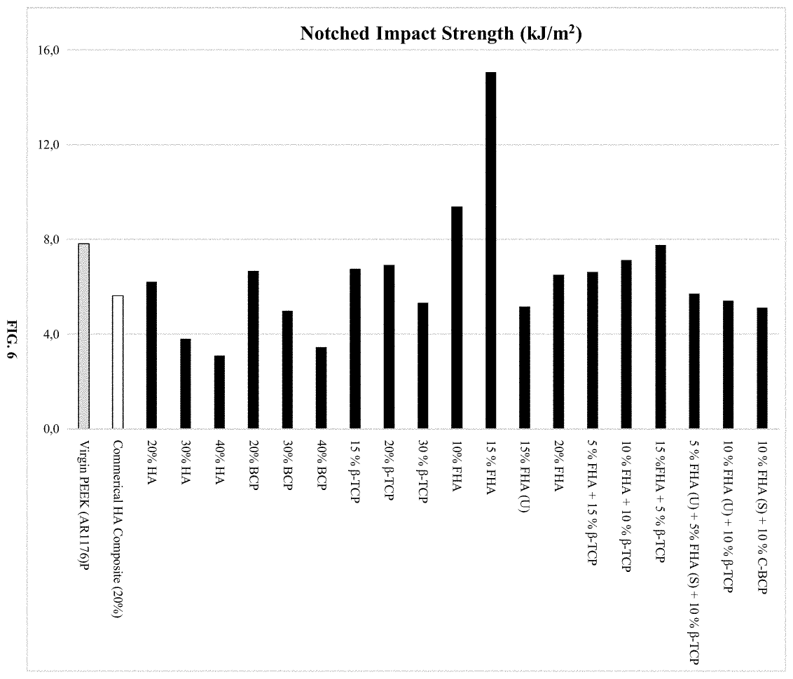

The present invention is also capable of producing composite materials which have enhanced tensile modulus and flexural modulus, which can be understood as the stiffness of a material. Referring now to FIG. 7, it can be seen that the presently disclosed compositions containing, for example, hydroxyapatite (HA), biphasic calcium phosphate, or fluorinated hydroxyapatite (FHA) have improved stiffness values compared to the virgin polyetheretherketone (PEEK) material. Further compositions of the presently disclosed invention have modulus values comparable to similar commercially available composite materials, but retain the flowability properties as described above and depicted in FIG. 2.

In further forms of the presently disclosed invention, not less than about 5% by weight and not more than about 45% by weight of the composition comprises the bioactive additive with the remaining constituent being a biocompatible polymeric material comprising of not less than about 55% by weight and not more than about 95% by weight. Other preferred compositions contain not less than about 15% by weight and not more than about 30% by weight of the composition comprising the bioactive additive.

One embodiment, but not limitation, of the herein disclosed invention comprises said bioactive additive comprising a ceramic capable of eliciting a biological response. Preferably, said biological response allows for cellular bonding at the surface of the herein disclosed composite compositions. The presently disclosed invention is able to promote both the formation of an apatite layer on the surface of the material as well as the attachment and proliferation of osteoblasts. In one embodiment, it displays the effect of submersing the presently disclosed inventive compositions in simulated body fluid and the resulting growth of apatite, an in vitro assessment and simulation of bone growth. In another embodiment, it shows the attachment and proliferation of osteoblast cells on the surface of the virgin polymeric material as well as the effects on attachment and proliferation of osteoblasts of the presently disclosed inventive compositions.

The above ceramic may be selected from a number of ceramics, including synthetic, natural, bioresorbable, or non-bioresorbable ceramics. Preferably, the ceramic is chosen from a type of calcium salt. Preferably, said calcium salt is a calcium phosphate, calcium sulfate, or calcium carbonate. In further embodiments, but not limitations, of the herein disclosed invention, the bioactive additive is selected from a metallic species such as magnesium, zinc, strontium, barium, or bismuth. Preferably, this metallic species is capable of eliciting a positive cellular response while also enhancing mechanical properties of said composition.

In further embodiments, the additive may be a ceramic that is prepared via hydrolysis. For example, hydrolysis prepared calcium phosphate, results in more rapid uptake of ions such as fluorine. This added potential for fluorine uptake is beneficial for the present invention as the doping of calcium salts with ionic species, for example, fluorine, is disclosed within the present inventive composition to be beneficial for mechanical, biological, and processing properties.

In further embodiments, the additive may be a ceramic that is preferably carbonated. For example, carbonated hydroxyapatite can be formed by the substitution of carbonate ions for hydroxyl ions within hydroxyapatite resulting in carbonated apatite.

Further, it is shown in the presently disclosed invention that carbonated hydroxyapatite, wherein between about 1% molar to about 6% molar of the hydroxyl groups are substituted with carbonate groups, is able to result in improved biological and processing properties.

In further embodiments, the additive may contain a dopant species. Preferably, the dopant species comprises no less than about 0.5% molar to no more than about 4% molar to the additive.

Further, the dopant species is preferably fluorine. Suitably, this fluorine may aid in the interfacial bonding between the bioactive additive and the polymeric matrix therefore enhancing the mechanical properties such as notched impact strength.

The compositions of the herein disclosed invention have a wide variety of applications. For example, the compositions may form composites that may be load-bearing and be used to form shaped medical implant articles such as intervertebral fusion cages. Other embodiments may also find advantageous use of the present invention wherein the application requires high-strength and/or high-ductility compositions for orthopaedic applications. For example, but not limitation, compositions may exist as shown in Table 2.

TABLE 2

| Composition | % wt. of Composition | | | | |
|---|---|---|---|---|---|
| Example | BCP | β-TCP | FHA | HA | C-BCP |
| 1 | 20 | | | | |
| 2 | 30 | | | | |
| 3 | 40 | | | | |
| 4 | | 15 | | | |
| 5 | | 20 | | | |
| 6 | | 30 | | | |
| 7 | | | 10 | | |
| 8 | | | 15 | | |
| 9 | | | 20 | | |
| 10 | | | | 20 | |
| 11 | | | | 30 | |
| 12 | | | | 40 | |
| 13 | | 15 | 5 | | |
| 14 | | 10 | 10 | | |
| 15 | | 5 | 15 | | |
| 16 | | | 10 | | 10 |

The processing of composite such as those disclosed herein may be fabricated through the use of mixing and blending. For example, in order to achieve improved dispersion of the additive throughout the polymeric material may be achieved by first physically blending the constituent powders through use of a rotational mixer. Subsequently, the physically blended additive and polymer may be processed through thermal methods such as the use of a twin-screw extruder. As an example of a potential method of processing through twin-screw extrusion, the heating zones may comprise those listed below but are not limited to the disclosed specific values in Table 3.

TABLE 3

| RPM | Heating Zones | | | | | | | | |
|---|---|---|---|---|---|---|---|---|---|
| 550 ± 25 | 1 | 2 | 3 | 4 | 5 | 6 | 7 | 8 | Die |
| Feed Rate (g/hr) | 23 ± 15° C. | 285 ± 30° C. | 365 ± 20° C. | 410 ± 20° C. | 410 ± 20° C. | 410 ± 20° C. | 410 ± 20° C. | 410 ± 20° C. | 410 ± 20° C. |
| 180 ± 20 | | | | | | | | | |

Further embodiments of the herein disclosed invention may encompass the fabrication of the herein presented compositions into net-shape or near-net shape articles for orthopaedic implants. The herein disclosed compositions can be readily injection molded and/or compression molded. The injection molding of a screw article can be accomplished due to the increased flowability of the presently disclosed invention. Further, the compression molding and subsequent machining of a load-bearing device, such as a spinal cage can also be achieved.

It can thus be understood from this teaching that the presently disclosed invention achieves novel compositions capable of enhancing the processability, shown by the flowability of the composites, as well as the mechanical and biological properties of composites.

The present invention describes a composition with novel properties in regards to strength, ductility, and flowability for the processing. This processing may be carried out in order to form, as an example but nota limitation, of orthopaedic medical devices such as, but not limited to, synthetic bone prosthesis, spinal cages, suture anchors, etc.

### DETAILED DESCRIPTION OF THE PREFERRED EMBODIMENTS

For the purpose of promoting a thorough understanding of the presently disclosed invention and not as a limitation of potential future embodiments of the herein disclosed invention, below are given examples of potential embodiments of the herein disclosed invention with reference made to specific embodiments with specific language being used to describe the same. It is therefore to be understood that no limitation of the scope of the herein disclosed invention shall be read into the preferred embodiment examples herein described or further modifications of the disclosed invention, with such further modifications and/or applications being those which would occur normally to one skilled in the art of the herein disclosed invention and related fields of study.

### EXAMPLE 1

The polyetheretherketone (PEEK), was compounded with 20% by weight hydroxyapatite (HA).

The mixture was compounded together by first blending the powders physically through the use of an inversion blender. The blended materials were then dried at a furnace temperature of 160° C. for 4 hours until moisture content reached less than 500 ppm as determined by use of a Computrac® Vapor Pro® Moisture Analyzer.

The physically blended, dried powders were then compounded together through thermal processing using a conventional melt extrusion apparatus. Under these conditions, the die temperature is maintained at 410° C. at which temperature the PEEK is molten and the HA is not changed morphologically. The resulting blend was extruded into forms for further processing such as molding, extruded stock shapes, etc.

### EXAMPLE 2

PEEK powder was compounded with 20% by weight biphasic calcium phosphate (BCP) in a manner similar to EXAMPLE 1.

The mixture was compounded together by first blending the powders physically through the use of an inversion blender. The blended materials were then dried at a furnace temperature of 160° C. for 4 hours until moisture content reached less than 500 ppm as determined by use of a Computrac® Vapor Pro® Moisture Analyzer.

The physically blended, dried powders were then compounded together through thermal processing using a conventional melt extrusion apparatus. Under these conditions, the die temperature is maintained at 410° C. at which temperature the PEEK is molten and the BCP is not changed morphologically. The resulting blend was extruded into forms for further processing such as molding, extruded stock shapes, etc.

### EXAMPLE 3

PEEK powder was compounded with 20% weight β-tricalcium phosphate (β-TCP) in a manner similar to EXAMPLE 1 and EXAMPLE 2.

The mixture was compounded together by first blending the powders physically through the use of an inversion blender. The blended materials were then dried at a furnace temperature of 160° C. for 4 hours until moisture content reached less than 500 ppm as determined by use of a Computrac® Vapor Pro® Moisture Analyzer.

The physically blended, dried powders were then compounded together through thermal processing using a conventional melt extrusion apparatus. Under these conditions, the die temperature is maintained at 410° C. at which temperature the PEEK is molten and the β-TCP is not changed morphologically. The resulting blend was extruded into forms for further processing such as molding, extruded stock shapes, etc.

### EXAMPLE 4

PEEK powder was compounded with 5% by weight FHA and 15% by weight β-tricalcium phosphate (β-TCP) in a manner similar to EXAMPLE 1, EXAMPLE 2, and EXAMPLE 3.

The mixture was compounded together by first blending the powders physically through the use of an inversion blender. The blended materials were then dried at a furnace temperature of 160° C. for 4 hours until moisture content reached less than 500 ppm as determined by use of a Computrac® Vapor Pro® Moisture Analyzer.

The physically blended, dried powders were then compounded together through thermal processing using a conventional melt extrusion apparatus. Under these conditions, the die temperature is maintained at 410° C. at which temperature the PEEK is molten and the FHA and β-TCP are not changed morphologically. The resulting blend was extruded into forms for further processing such as molding, extruded stock shapes, etc.

### EXAMPLE 5

According to the following protocol, the injection molding of the herein disclosed polymeric material as well as polymeric composite materials was performed.

Compounded material containing samples of the herein disclosed inventive compositions were dried at a furnace temperature of 160° C. for 4 hours. Once the moisture content of the dried material reached less than 500 ppm, the material was injection molded. The nozzle temperature was maintained at 360° C. with the mold temperature being maintained at 180° C.

### EXAMPLE 6

According to the following protocol, the compression molding of the herein disclosed polymeric composite materials was performed.

The composition in either the form of blended powder or an extruded composite pellet was dried first 3 hours at a furnace temperature of 150° C., otherwise, overnight at 120° C. A small amount of polytetrafluoroethylene (2-3%) was found to be a beneficial addition to the compression mold for easier de-molding but is not required. Once the material to be molded has settled into the mold to allow powder packing, the heated platens, both at a temperature of 420° C. are pressed together. The cooling rate is then controlled to be 40° C./ hr. Once the platen temperatures reached 140° C., the article was able to be demolded.

### EXAMPLE 6

According to the following protocol, the tensile properties of the herein disclosed polymeric material as well as polymeric composite materials were determined.

In accordance with ISO 527, dog-bone samples were loaded into a Zwick Z020 Retroline with a 20 kN load cell to determine tensile strength of the dog-bone samples. Injection molded dog-bone samples were tested at a room temperature of 23° C. Dog-bone samples were placed into clamps at a 20 mm gauge length and loaded with a constant speed of 5 mm/min until failure of the sample. Tensile modulus were tested in the same manner with the load speed changing to 1 mm/min.

### EXAMPLE 7

According to the following protocol, the flexural properties of the herein disclosed polymeric material as well as polymeric composite materials were determined.

In accordance with ISO 178, flexural bar samples were loaded into a Zwick 1445 with a 500 N load cell to determine flexural properties of the flexural bar samples. Injection molded flexural bar samples were tested at a room temperature of 23° C. Flexural bar samples were placed onto a 63±5 mm span and with a loading nose of 5±0.2 mm and support noses of 5±0.2 mm and loaded with a constant speed of 2 mm/min until failure of the sample.

### Example 8

According to the following protocol, the impact strength of the herein disclosed polymeric material as well as polymeric composite materials was determined.

In accordance with ISO 180, notched impact samples were fabricated into with a notch radius of 0.25±0.05 mm, a notch root of 8±0.2 mm, and a notch angle of 45±1° . The notched impact samples loaded into a Zwick HIT 5.5 P with a 1 J pendulum to determine tensile strength of the dog-bone samples. The notched impact samples were then tested at a room temperature of 23° C.±5° C.

### EXAMPLE 9

According to the following protocol, the flowability of the herein disclosed polymeric material as well as polymeric composite materials was determined.

In accordance with ISO 1133, flowability samples were taken in pellet form and loaded into a Göttfert 3010. The temperature of the testing zone was raised to 395° C.±15° C. and held for the duration of the test. A 5 kg mass as placed above the melt zone in order to force the melt from the heating zone. The flowability was then recorded as the melt volume flow rate by determining the mass of the sample that flowed after 10 minutes.

### EXAMPLE 10

A sample of the herein disclosed compositions was injection molded into an interference screw design such as those commonly used in orthopaedic implants.

### EXAMPLE 11

A sample of the herein disclosed compositions was extruded into a stock-shape and subsequently machined to a spinal cage design such as those commonly used in orthopaedic implants.

Although the compositions have been listed here in regards to their compositional makeup, further processing of the produced pellets may be carried out to fabricate devices such as medical implants which require superior mechanical properties and the ability to facilitate bone growth.

It is clear that numerous modifications and variations of the present invention are possible in light of the above teaching and it is therefore to be understood that within the scope of the appended claims, the invention may be practiced otherwise than as specifically described herein.

### EXAMPLE 12

### Filament Fabrication

Filaments of the composite material were fabricated through use of a twin-screw extruder utilizing the previously stated drying, blending, and compounding protocols. The bioinert polymer (PEEK) was blended with a bioactive added calcium phosphate derivative through a dry blending procedure followed by thermal processing via twin-screw compounding. First, the respective materials were weighed out in powder form to comprise a total compositional ratio of 80 wt % polymer (80g PEEK) and 20 wt % biphasic calcium phosphate (20 g calcium phosphate derivate powder) using an inversion blender. The blended powders were then dried overnight at 160° C. After drying the powder blend was processed in a twin-screw compounder with temperature profiles between 250° C. and 410° C. The resultant extrudate was then drawn into a composite filament of diameter of 1.7+/−0.1 mm and collected for use in fused filament fabrication.

### EXAMPLE 13

### Fused Filament Fabrication of Samples

The PEEK +20% nBCP constructs were printed using the 3NTR A4v3 fused filament fabrication printer. The constructs were printed using a nozzle temperature of 410° C., a bed temperature of 135° C., and a chamber temperature of 75 ° C. The first layer of the construct was printed ata thickness of 0.4 mm while the rest of the part was printed at a layer height of 0.1 mm. A raft was used for each print to prevent the parts from warping and to better adhere to the build plate. This procedure was carried out to fabricate all composite testing samples with the variation existing only in the geometries of pore sizes as selected. Samples of virgin PEEK materials were printed in a similar fashion but required an elevated nozzle temperature of 430° C., chamber and build plate temperature of 200° C. using a Meditool printer.

### EXAMPLE 14

### Thermogravimetric Analysis of Filament

Thermogravimetric analysis was carried out on the composite filament to quantify weight percentage of the bioactive additive, biphasic calcium phosphate, in the composite material. Approximately 25 mg of sample material was placed into a TGA Q500 under an air atmosphere and exposed to a temperature profile as follows: equilibration at 25° C. for at least 5 minutes followed by a temperature ramp at 10° C./min up to 900° C. and then held isothermally for 5 minutes. The reported remaining mass was taken as the amount of bioactive additive within the composite material.

### EXAMPLE 15

### Mechanical Characterization of 3D Printed Samples

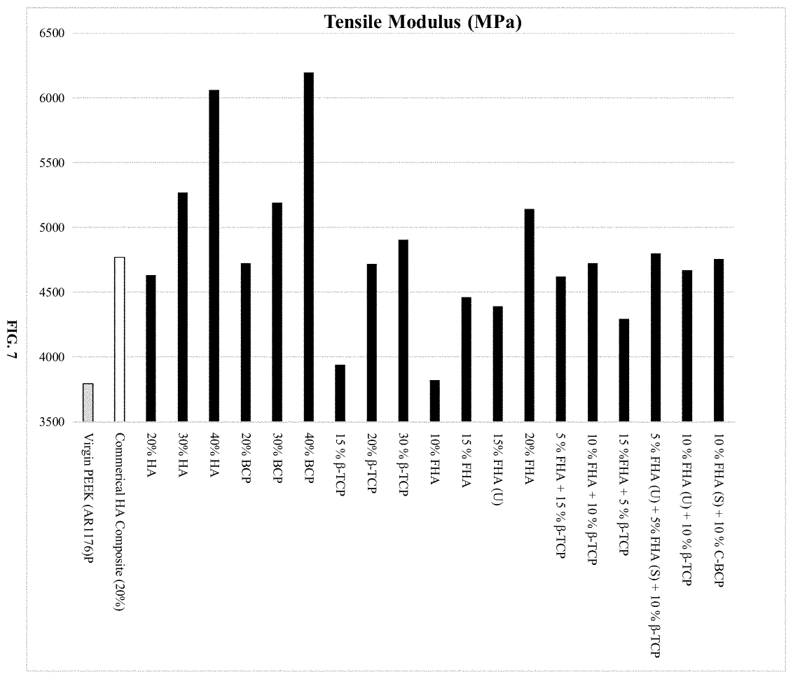

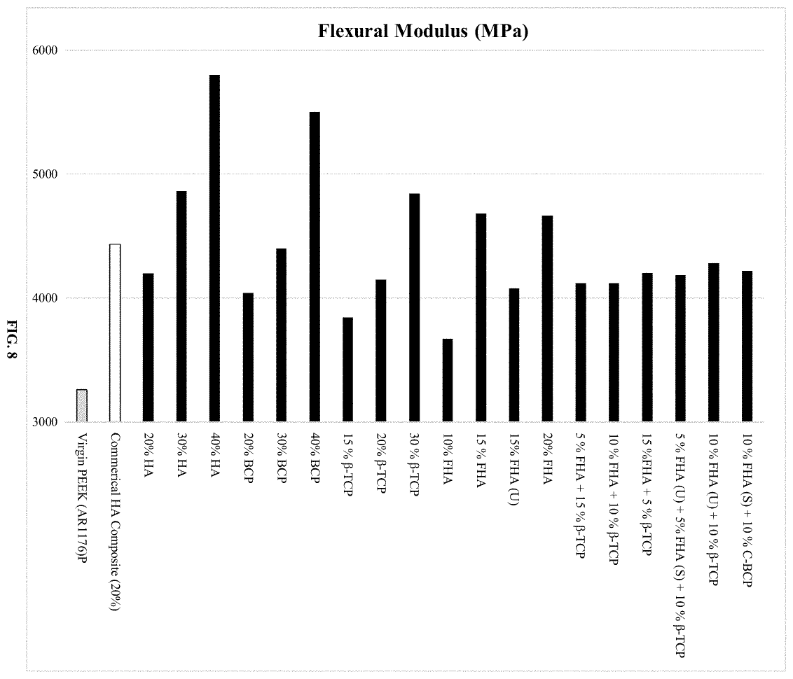

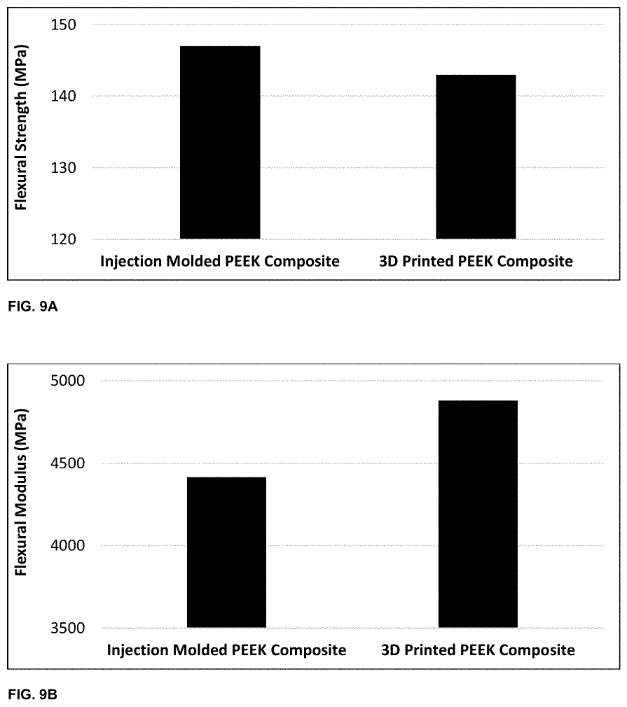

Tensile testing of the 3D printed samples was carried out in accordance with ISO 527 as follows. After preparing the tensile samples via either injection molding or fused filament fabrication (3D printing), the samples were loaded into an Instron 3366 and subjected to an initial strain rate of 0.2 mm/min which was then increased to 5 mm/min. Properties including the modulus, stress at yield, stress at maximum load, and elongation at break (FIG. 10A, FIG. 10B) were measured and reported. Similarly, flexural tests were carried out on both injection molded and 3D printed samples (FIG. 9A, FIG. 9B). In accordance to ISO 178 (2010), samples with span lengths of 64mm were exposed to a strain rate of 2 mm/min to failure. The flexural strength, flexural modulus, and flexural strain at break were measured and compared (FIG. 9A, FIG. 9B).

### EXAMPLE 16

### Compression Testing of Medical Device Prototype

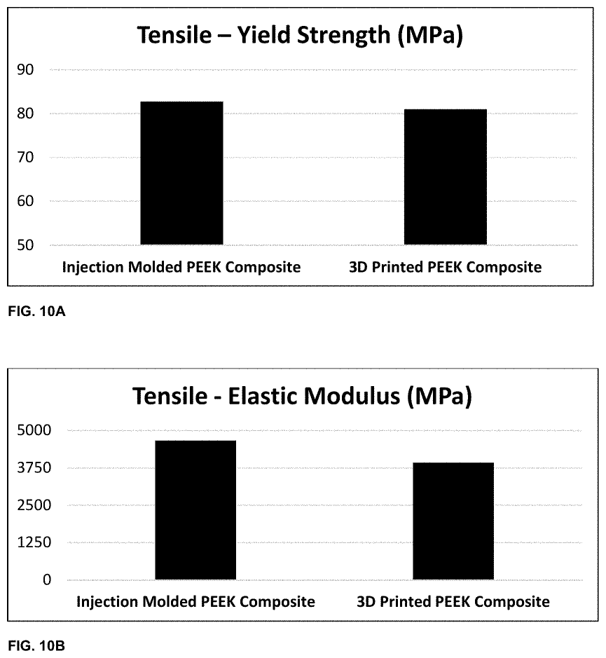

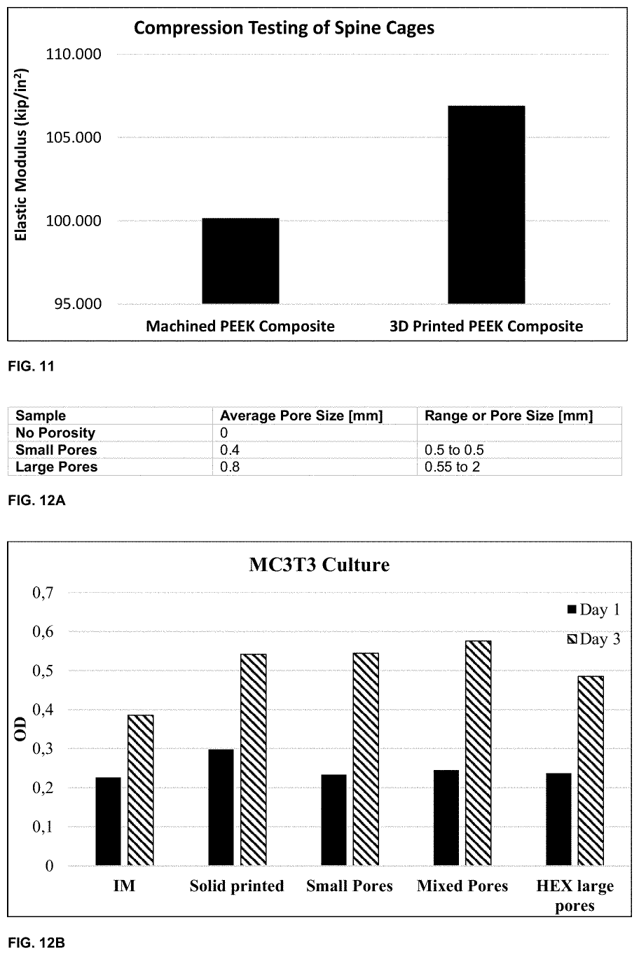

As depicted in FIG. 11, compression testing of the 3D printed spinal fusion cages was performed as suggested by ASTM F2077 with minor modifications. Aluminum plates were first machined as a negative space for fitting of the spinal cages to ensure equivalent load distribution across to testing surface. After placing the cages into the aluminum fixture, the cages were exposed to an increasing compressive load at a rate of 1 mm/min axially to mimic the load which would be found in an implantation environment. The elastic modulus and load to failure were measured and compared for machined and 3D printed devices.

### EXAMPLE 17

### MTS Assay

To investigate the effects of fabrication techniques on cellular viability, attachment, and proliferation, samples of PEEK Composite were fabricated through injection molding as well as fused filament fabrication (3D Printed). The 3D printed samples were also fabricated with varied degrees of porosity and multiple pore sizes as depicted in FIG. 12A in the processing method described previously. 3D printed PEEK specimens were disinfected with 70% alcohol and autoclaved prior to cell seeding. MC3T3-E1 cells (ATCC, Manassas, Va.) were plated above the polymers in a 24-well plate at a concentration of 4x10⁴ per well. After 24 hours of incubation at 37° C. in Alpha- MEM media+10% FBS (Invitrogen/ThermoFisher, Carlsbad, Calif.), the polymers were transferred to new 24-well plates for further incubation or for assays. Manufacturer instructions were followed for the LIVE/DEAD viability/cytotoxicity assay (Invitrogen/ThermoFisher) and Celltiter 96 Aqueous One Solution (MTS) assay (Promega, Madison, WI). The assays were done after 24 and 72 hours of incubation. Confocal microscopy was performed using an LSM 780 (Zeiss) microscope. FIG. 12B depicts cells maintained their viability on the surface of 3D Printed PEEK Composite similar to injection molded PEEK Composite surfaces. The 3D Printed PEEK Composite surface provided more surface area for cell growth.

1. An orthopaedic, bioactive composition with enhanced flowability and enhanced ductility, comprising a composite comprising:
a. a high-viscosity, biocompatible polymeric matrix; and
b. a homogenously distributed additive.
wherein said polymeric matrix comprises a polyaryletherketone (PAEK), more preferably polyetheretherketone (PEEK), and wherein said additive comprises an additive species for improved melt-processability, article mechanical properties, and biological activity.

2. The composition of claim 1, wherein said polymeric matrix comprises a powder before processing and comprises an average diameter of between about 5 μm to about 2,500 μm, more preferably 10 μm to about 1,000 μm, most preferably 50 μm to about 500 μm.

3. The composition of claim 2, wherein said powder has a density of about 1.15 g/cm3 to about 1.5 g/cm3.

4. The composition of claim 1, wherein said polymeric material has a melt volume flow rate (MVR) of about 13 cm³/10 min to about 15 cm³/10 min at 400° C. and 5 kg of mass.

5. The composition of claim 1, wherein said polymeric matrix has an elongation to failure of about 55% to about 70%.

6. The composition of claim 1, wherein said additive acts as a flowing-aid.

7. The composition of claim 1, wherein said additive comprises a particulate with an average particulate size of between 0.2 μm to 1.5 μm, more preferably 0.35 μm to 1.2 μm, most preferably 0.5 μ to 1.0 μm.

8. The composition of claim 1, wherein said additive comprises a calcium salt.

9. The composition of claim 8, wherein said calcium salt is hydrolyzed or carbonated.

10. (canceled)

11. The composition of claim 8, wherein said calcium salt comprises a salt selected from the group consisting of calcium phosphate, calcium sulfate, and calcium carbonate.

12. The composition of claim 1, wherein said additive comprises tricalcium phosphate, and wherein said tricalcium phosphate comprises β-tricalcium phosphate.

13. (canceled)

14. The composition of claim 1, wherein said additive comprises biphasic calcium phosphate, and wherein said biphasic calcium phosphate contains a blend of hydroxyapatite and tricalcium phosphate in a ratio of 20:80, more preferably 40:60, most preferably 80:20.

15. (canceled)

16. The composition of claim 1, wherein said additive comprises a weight ratio of 1% to 35%, more preferably 10% to 30%, most preferably 15% to 25% by weight of the said composition.

17. The composition of claim 1, wherein said composite has a melt volume flow rate of about 9 cm3/10 min to about 12 cm3/10 min at 400 ° C. and 5 kg of mass.

18. The composition of claim 1, wherein said composite has an elongation to failure of about 25% to about 50%.

19. An orthopaedic, bioactive composition with enhanced strength, comprising a composite comprising:
a. a high-strength, biocompatible polymeric matrix; and
b. a homogenously distributed, dopant-containing bioactive additive.
wherein said polymeric matrix comprises a polyaryletherketone (PAEK), more preferably polyetheretherketone (PEEK), and wherein said dopant-containing bioactive additive comprises an additive species for improved melt-processability, article mechanical properties, and biological activity.

20. The composition of claim 19, wherein said polymeric matrix comprises a powder before processing and comprises an average diameter of between about 5 μm to about 2,500 μm, more preferably 10 μm to about 1,000 μm, most preferably 50 μm to about 500 μm.

21. The composition of claim 20, wherein said powder has a density of about 1.12 g/cm3 to about 1.5 g/cm3.

22. The composition of claim 19, wherein said polymeric matrix has a tensile strength of about 88 MPa to about 92 MPa.

23. The composition of claim 19, wherein said dopant is selected from the group consisting of fluorine, magnesium, zinc, strontium, barium, and bismuth.

24. (canceled)

25. The composition of claim 19, wherein said dopant comprises between about 1% molar to about 5% molar of said additive.

26. (canceled)

27. The composition of claim 19, wherein said dopant-containing bioactive additive comprises an average particulate size of between 0.2 μm to 1.5 μm, more preferably 0.35 μm to 1.2 μm, most preferably 0.5 μm to 1.0 μm.

28. The composition of claim 19, wherein said dopant-containing bioactive additive comprises a calcium salt.

29. The composition of claim 28, wherein said calcium salt is hydrolyzed or carbonated.

30. (canceled)

31. The composition of claim 28, wherein said calcium salt comprises a salt selected from the group consisting of calcium phosphate, calcium sulfate, and calcium carbonate.

32. The composition of claim 19, wherein said dopant-containing bioactive additive comprises fluorinated calcium phosphate.

33. The composition of claim 32, wherein said fluorinated calcium phosphate comprises fluorapatite.

34. The composition of claim 19, wherein said dopant-containing bioactive additive comprises about 1% to 35%, more preferably 10% to 30%, most preferably 15% to 25% by weight of the said composition.

35. The composition of claim 19, wherein said composite has a tensile strength of about 85 MPa to about 105MPa.

36-89. (canceled)
